(19) United States
(12) Patent Application Publication
(10) Pub. No.: US 2024/0359030 A1
(43) Pub. Date: Oct. 31, 2024

(54) CASING FOR DERMATOLOGICAL TREATMENT DEVICE

(71) Applicant: URGO RECHERCHE INNOVATION ET DEVELOPPEMENT, Chenove (FR)

(72) Inventors: Manuelle BAHUON, Dijon (FR); Jonathan BRICE, Beaune (FR); Valentin FLORIOT, Longvic (FR); Fanny GUINOT-SKRZYPSKI, Velars sur Ouche (FR); Michel LAMOISE, Pouxeux (FR)

(21) Appl. No.: 18/686,667

(22) PCT Filed: Sep. 5, 2022
(86) PCT No.: PCT/EP2022/074612

§ 371 Date: Feb. 26, 2024

(30) Foreign Application Priority Data
Sep. 8, 2021 (FR) 2109398

(51) Int. Cl. A61N 5/06; A61N 5/067

(52) U.S. Cl. CPC A61N 5/0616; A61N 5/067; A61N 2005/0626; A61N 2005/0632; A61N 2005/0644

(57) ABSTRACT

This relates to a protective casing for a dermatological treatment device that includes a laser, wherein the casing has an elongated shape that envelopes the device and forms at one end a head including an opening capable of allowing a beam fired by the laser to pass; the casing including several parts assembled together, where the head is formed from a single, part.

This patent application claims the priority of patent application FR 2109398 filed on Sep. 8, 2021, which is incorporated into this patent application by reference.

### FIELD OF THE INVENTION

The present invention relates to a dermatological treatment device comprising a laser head capable of firing a laser beam. Such a device is typically used to heat a patient's target area comprising dermal tissues in order to accelerate the healing thereof. In particular, the present invention relates to the casing of this device.

### PRIOR ART

It is well known that, when producing such a device, all the functional elements (laser, control unit, pyrometer, ventilation, battery power supply, etc.) are housed in a plastic casing. This casing then protects said functional elements against potential damage or contamination. Furthermore, its shape allows for it to be handled by an operator in such a way as to enable the proper utilization of said device.

The head of the device comprising the opening through which the laser beam passes is intended to be brought in close proximity to the skin of the patient to be treated. Thus, it is therefore possible for the head to accidentally come into contact with patients' bodily fluids (blood, lymph, etc.), which are potentially contaminants.

A plastic casing is conventionally manufactured in two longitudinal halves, relative to a plane of symmetry passing through the device along a longitudinal axis that essentially matches the laser firing axis. Also, the seam between the two halves, at the very end of the head of the device, noticeably occurs in an area that can come into contact with the patient's skin.

A seal in the joint between the two halves is intended to block liquid from penetrating and thus prevent any internal contamination of the device. However, it has been observed that some devices have had liquid penetrate their casings' interiors, which can potentially lead to the contamination of patients or personnel in contact with the laser treatment device.

Therefore, the present invention is intended to overcome this drawback by preventing any risk of internal contamination of the casing.

### SUMMARY OF THE INVENTION

Thus, the object of the invention is a protective casing for a dermatological treatment device comprising a laser; said casing having an elongated shape that envelopes the device and forms, at one end, a head comprising an opening that allows for the passage of a beam fired by the laser; said casing comprises several parts assembled together, wherein the head is made up of a single part.

Particular characteristics or embodiments, usable alone or in combination, include the following:
The casing has an air inlet grille and an air outlet grille made of the same material as the casing itself (that is to say they are manufactured as a seamless part of the casing).
Said air inlet grille is positioned near the end of the casing in the part forming the head.
Said air outlet grille is positioned near the other end of the casing, opposite the head, and whose plane is preferably not parallel to the surface of the casing.
The casing also comprises a window through which a pyrometer can operate, wherein said window is positioned away from the outer surface of the casing and is deep-set into the casing itself and/or surrounded by a protruding ring.
The casing further comprises a light guide that is made of translucent material and bisects a small section of the casing (preferably partially encircling at least 180° of the exterior of the casing) in order for at least one status indicator for the device to be visible externally.
The light guide is inserted between the part forming the head and the rest of the casing.
The casing also includes an access panel to programming ports.
The casing further comprises at least one laser firing button, where said button(s) form a protuberance relative to the surface of the casing.
The different parts are joined together by screws, where each screw is deep-set into the plastic and the subsequent hole is covered by a screw cover.

A second object of the invention relates to a dermatological treatment device comprising a laser head capable of firing a laser beam towards a target area comprising a patient's dermal tissues in order to accelerate healing, at least one pyrometer near said laser head that is capable of measuring the temperature of the target area, at least one fan capable of dissipating the heat released by said laser, at least one means of monitoring capable of controlling at least one laser beam inhibition means as soon as the temperature measured by the pyrometer exceeds a threshold value, said device further comprises a casing as defined above.

Such a device is described in detail in the international applications WO 2011/080574 and WO2017/118606.

Preferably, such a device would further comprise a protective sleeve such as described in the international application WO 2017/118605.

### BRIEF DESCRIPTION OF THE DRAWINGS

The invention may be better understood upon reading the following description, given solely by way of example, and referring to the appended figures in which:

FIG. 1 is a perspective view of the entire casing.

FIG. 2 is an exploded view of the perspective view of FIG. 1.

FIG. 3 is a perspective view of the part comprising the head.

FIG. 4 shows in detail the window for the pyrometer.

FIG. 5 is a perspective view of a light guide.

FIG. 6 is a perspective view of an access panel.

### DETAILED DESCRIPTION OF THE EMBODIMENTS

The invention relates to a protective casing 1 for a dermatological treatment device comprising a laser capable of firing a laser beam. Such a device is typically used to heat a targeted area comprising the dermal tissues of a patient in order to accelerate healing.

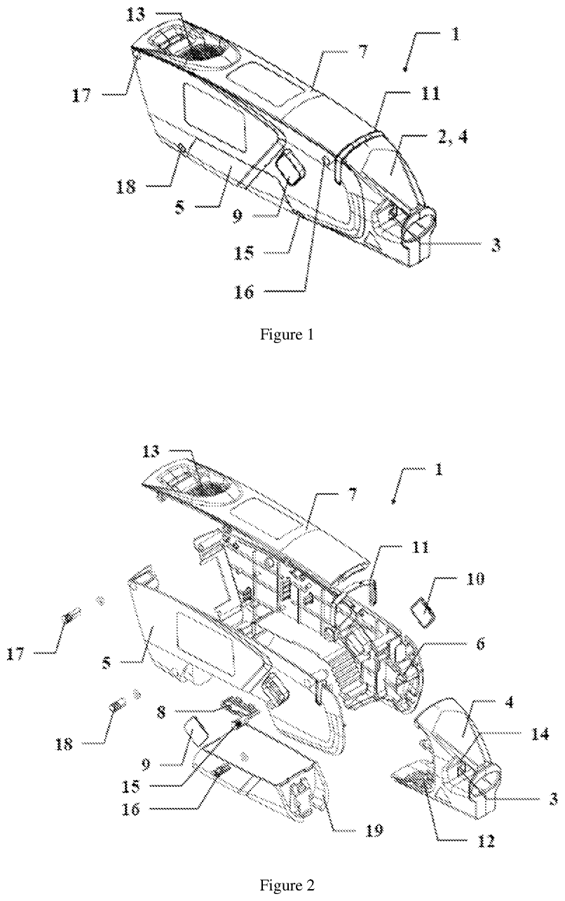

As illustrated in FIGS. 1 and 2, said casing 1 comprises multiple parts 4-11 assembled together so as to envelop the device. The casing 1 has an elongated oblong cigar-like shape. One of end of said casing 1 forms a head 2 where an opening 3 is placed that allows for the passage of a laser beam fired by the laser.

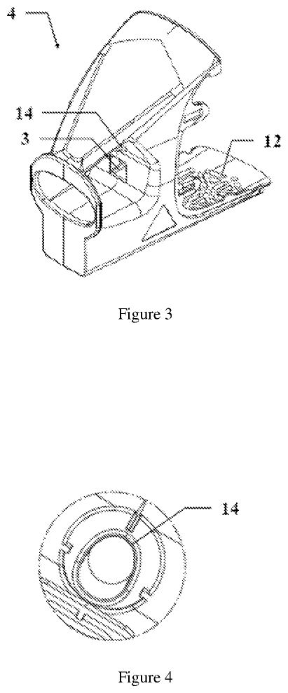

According to one characteristic, the head 2 of the casing 1 is made up of a single part 4 in order to eliminate the unfavorable characteristic of the prior art where a joint between two casing parts passes through the head 2, not far from the optical axis of the laser. An example of an embodiment of such a part 4 is illustrated in FIG. 3. Said part 4 thus forms the head 2 of the casing 1 without presenting a joint near the opening for the laser beam 3, since said joint could potentially come into contact with the patient and is therefore potentially subject to contamination.

The casing 1 itself is handheld: it constitutes a handle for gripping and operating the device during its utilization by an operator.

In addition to part 4 encompassing the head 2, according to the illustrated embodiment, the casing 1 further comprises a right half 5 and a left half 6 each forming, essentially symmetrically, one of the lateral halves of the casing 1. In the upper portion, a cover 7 is positioned between the two halves 5, 6. All parts 4-11, preferably made from plastic, are manufactured via molding. The casing 1 can also include a removable battery compartment 19.

The laser produces heat and therefore requires a means of cooling. Thus, the device incorporates a fan (not shown), capable of circulating air inside the casing and around the laser transmitter, in order to cool it. This requires an air inlet and outlet. It is well-known to include an air inlet grille and an air outlet grille in the casing, with the air passing from the inlet to the outlet. According to the prior art, metal grates are fixed on the casing in front of the inlet and outlet.

According to another characteristic, an air inlet grille 12 is formed directly in the casing 1 by means of holes. Advantageously, this allows for the fabrication of said air inlet grille using the same material as the casing 1 itself as part of the molding process. This reduces costs by eliminating a part and its associated assembly and fabrication processes. Analogously, an air outlet grille 13 is made from the same material as the casing 1. The grille itself can take any possible shape. The example illustrated shows grilles roughly shaped like a snowflake.

According to another characteristic, the air inlet grille 12 is positioned near the end of the casing 1 within the part 4 forming the head 2. As illustrated more particularly in FIG. 3, the air inlet grille 12 is positioned at the base of the part 4 forming the head 2, essentially directly below the laser transmitter (not shown). It is positioned far enough away from the opening for the laser 3 and the area contacting the skin so as not to risk contamination.

According to another characteristic, the air outlet grille 13 is positioned near the other end of the casing 1, opposite the head 2. This is shown in greater detail in FIG. 2.

This orientation of the two ventilation grilles 12, 13 allows for the circulating air-produced by the fan—to cool the entire volume between said ventilation grilles 12, 13, and therefore essentially the entire internal volume of the casing 1.

According to another characteristic, the air outlet grille 13 is positioned in such a way that the plane it occupies relative to the casing 1 is not parallel to the surface of said casing 1 at its location. This configuration was observed to permit the orientation of the air outlet grille 13 to be essentially parallel with the fan and thus improving both the efficiency of air circulation and, consequently, the operating efficiency of the device as a whole.

The device may also include a pyrometer. Said pyrometer is intended to measure the temperature of the patient's skin in the device's target area during treatment to ensure that overheating of said area does not occur. Also, near the opening 3 for the laser beam, the casing 1 includes a measuring window 14 through for the pyrometer. Said window 14 includes filter closing it while allowing measurement by the pyrometer.

It became apparent, in devices according to the prior art, that the operator could accidentally put his finger on said filter, thereby blocking the pyrometer window 14. This could have the effect, via the depositing of sebum or bodily fluids (blood, lymph, etc.) from the patient being treated, of obscuring the window and altering the temperature measurement (underestimation). However, the device according to the invention includes a control unit that restricts the power of the laser in accordance with the measured temperature. Thus, a fingerprint on the pyrometer window's 14 filter could potentially cause an overheating, or even burning, of the patient's skin.

Consequently, according to another characteristic, the casing 1 is shaped so that said window's 14 filter cannot come into contact with a finger. To accomplish this, the window 14 is set back from the exterior surface of casing 1. Said window 14 is thus located at the bottom of a hole. Either alternatively or complementarily, it is surrounded by a protruding ring. Due to its diameter being significantly smaller than that of a finger, said protrusion thus permits the exterior surface of the filter to be protected by preventing a finger from coming into contact with said filter positioned in the window 14. These characteristics are illustrated in the more detailed FIG. 4.

The device also includes at least one luminous status indicator, preferably an LED. Such an indicator can indicate the power status, laser operation status, overheating warning, low battery warning, etc.

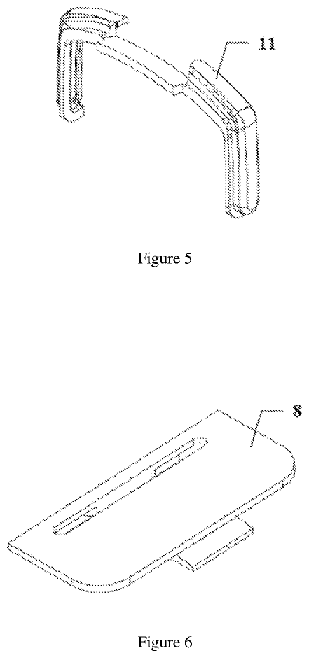

In order for said indicator light positioned inside the casing 1 to be visible from the outside, typically to an operator, the casing 1 comprises at least one area made of translucent material, preferably transparent. According to another characteristic, the casing 1 further comprises a light guide 11, made of translucent material, preferably transparent. Said guide 11 is shaped so as to form a small cross-section that partially encircles the elongated shape of the casing 1, i.e. a section transverse to the longitudinal axis (coincident with the optical axis of the laser) of the device. Said section preferably partially encircles at least 180°—more preferably at least 300°—of the casing's exterior, thereby permitting what resembles at least one status indicator of the device to be externally visible, over a corresponding angle of at least 180°, respectively at least 300°. A light guide according to a particular embodiment is illustrated in FIG. 5.

The light guide 11 is one of the parts 4-11 comprising the casing 1. According to another characteristic, the light guide 11 is inserted between the part 4 forming the head 2 and the remaining parts 5-10 of said casing 1. According to the illustrated embodiment, the light guide 11 is inserted between the part 4, the side halves 5, 6 and the cover 7. Said guide's complementary integration with the adjacent parts ensures its incorporation into the casing 1 without any other means of fixation.

It has been shown that the device includes a control unit. Advantageously, said control unit is programmable. In order to have this capacity, it has at least one port allowing it to be connected to a programming unit, in particular, in order to carry out software updates. Access to at least one of said programming ports is reserved for maintenance personnel and does not have to be accessible to the operator of the device. Also, according to the prior art, access to the programming connectors was achieved by dismantling the entire casing 1. This can be tedious for maintenance personnel.

Therefore, according to another characteristic, the casing 1 also includes an access panel 8 for the programming port(s). Said panel 8 is illustrated in greater detail in FIG. 5. Its insertion into the casing 1 is illustrated in FIG. 2.

In order to limit accessibility to operators and reserve access to maintenance personnel, the panel 8 is advantageously protected by a tamper-evident label.

In order to control the firing of the laser of the device, it includes at least one firing button. This at least one button is replicated on the casing by as many buttons 9, 10. Advantageously, the control of the laser's firing is secured by a dual pushbutton command: both buttons 9, 10 must be activated simultaneously to fire the laser. The mirrored positioning of the two buttons 9, 10, essentially along the same axis on opposite sides of the casing 1 (a right button 9 on the right half 5 and a left button 10 on the left half 6), guarantees that the thumb and the index finger are each placed on one of the buttons 9, 10, thereby ensuring that the operator's hand is properly gripping the casing 1.

According to another characteristic, said at least one button 9, 10 forms a protuberance relative to the surface of the casing 1. It has been demonstrated that this configuration allows the operator's fingers to be positioned naturally—in a haptic manner—on the buttons 9, 10 on the casing 1. This haptic effect could be further enhanced by incorporating a textured surface on said at least one button 9, 10 in order to better contrast with the smooth surface of the surrounding the casing 1. This difference in texture can be clearly felt with a finger. Such a touch-based configuration is particularly advantageous in positioning the finger on the button 9, 10 because the device is associated with a protective cover and the operator wear gloves, both of which may alter the grip of the device.

The casing 1 is fabricated by assembling the different parts 4-11. Said parts 4-11 are mainly held together by screws. In the illustrated embodiment, four screws secure the right half 5 to the left half 6 of the casing 1. Brackets in part 4 and in cover 7 are positioned so that certain ones of said screws will pass through them and thus assemble part 4 and cover 7 to the two halves 5, 6.

According to another characteristic, each of the screws is recessed into the casing, so that the screw head is located below the surface of the casing 1. These holes are then hidden by screw covers 15-18. These screw covers 15-18, in that they close the hole and present a continuous surface flush with the surface of the casing 1, reduces the gaps in said casing and thus improves both its impermeability and resistance to contamination. The screw covers 15-18 are typically screwed into the holes via quarter turn thread. In addition, said screw covers 15-18 improve the aesthetics of the casing 1.

The invention has been illustrated and described in detail in the drawings and the preceding description. These should be considered illustrative and given by way of example and not as limiting the invention to this description alone. Numerous embodiment variants are possible.

### LIST OF REFERENCE TERMS

1: Casing
2: Hcad
3: Opening for laser
4: Part forming the head
5: Right half
6: Left half
7: Cover
8: Access panel
9: Right button
10: Left button
11: Light guide
12: Inlet air grille
13: Outlet air grille
14: Window for pyrometer
15-18: Screw cover
19: Battery compartment

1. A protective casing for a dermatological treatment device comprising a laser, said casing having an elongated shape that envelopes the device, forming at one end a head comprising an opening capable of allowing a beam fired by the laser to pass and comprising an area of contact with the skin, said casing comprising several parts assembled together, wherein the head the head is made up of a single part.

2. The casing according to claim 1, further comprises an air inlet grille and an air outlet grille which are part of and made from the same material as the casing itself.

3. The casing according to claim 2, wherein the air inlet grille is positioned near the end in the part forming the head and the air outlet grille is positioned near the other end of the casing opposite the head.

4. The casing according to claim 1, wherein the casing further comprises a measurement window for a pyrometer, said window being set back from the exterior surface of the casing, recessed into the casing itself and/or surrounded by a protruding ring.

5. The casing according to claim 1, further comprising a light guide made of a small piece of translucent material and partially encircling the casing in order to have at least one device status indicator externally visible.

6. The casing according to claim 5, where the light guide is positioned between the part forming the head and the rest of the casing.

7. The casing according to claim 1, further comprising an access panel to programming port(s).

8. The casing according to claim 1, further comprising at least one laser firing button, wherein said at least one button forms a protuberance relative to the surface of the casing.

9. The casing according to claim 8, wherein at least one button of the casing has a textured surface.

10. A dermatological treatment device comprising a laser head capable of firing a laser beam towards a target area of a patient comprising dermal tissues in order to accelerate healing, at least one pyrometer near the laser head and capable of measuring the temperature of said target area, at least one fan capable of dissipating the heat released by the laser head, at least one means of monitoring capable of controlling at least one laser beam inhibition means as soon as said temperature exceeds a threshold value, wherein the dermatological treatment device further comprises the casing of claim 1.

11. The casing according to claim 3, wherein the casing further comprises a measurement window for a pyrometer, said window being set back from the exterior surface of the casing, recessed into the casing itself and/or surrounded by a protruding ring.

12. The casing according to claim 11, further comprising a light guide made of a small piece of translucent material and partially encircling the casing in order to have at least one device status indicator externally visible.

13. The casing according to claim 12, where the light guide is positioned between the part forming the head and the rest of the casing.

14. The casing according to claim 13, further comprising an access panel to programming port(s).

15. The casing according to claim 14, further comprising at least one laser firing button, wherein said at least one button forms a protuberance relative to the surface of the casing.

16. The casing according to claim 15, wherein at least one button of the casing has a textured surface.
